(19) United States
(12) Patent Application Publication
(10) Pub. No.: US 2008/0319360 A1
(43) Pub. Date: Dec. 25, 2008

(54) Joint Device Enclosure

(76) Inventor: Abby Zukowski, Leonia, NJ (US)

Correspondence Address: Abby Zukowski, 540 Nordhoff Drive, Leonia, NJ, 07605, US

(21) Appl. No.: 11/765,454

(22) Filed: Jun. 20, 2007

(51) Int. Cl. A61F 5/40; A61F 13/00

(52) U.S. Cl. 602/2; 602/41

(57) ABSTRACT

An enclosure of which is created to comprise the inner layer areas of a joint device. The enclosure is specifically created and formed to receive within its open area a tempered therapeutic temperature generating material and or a support cushion padding type of material. The enclosure of the present invention is created in a unique and functional manner due primarily to its characteristic implemental methods of versatility as will be clearly specified and illustrated within the specifications of this application.

### BACKGROUND

The present invention relates to an enclosure of which its material composition is comprised of a breathable, porous and moisture resistant type of material. The joint devices presently offered do not effectively provide the needed optional qualities of tempered therapeutic treatment and of providing additional support to the joint in such an accommodating manner that is simple, efficient, and easy to use. Prior art devices possess complex, costly and user unfriendly prerequisites. In a few instances, the devices offered today are simply ineffective in their overall therapeutic performance. The modern individual demands simplicity, cost-efficiency, and overall effectiveness to be integrated trait characteristics of their joint devices of choice. An important aspect of the present invention is that it allows the use of a tempered therapeutic temperature generating material and a cushion support type of padding material to be efficiently and effectively implemented integrally throughout the enclosure of the present invention, by allowing the optional supplemental material to be strategically placed within the enclosure of which is located in the areas of the inner layers of a joint device. Consequently, this provides a truly unique and effective manner of implementing and providing effective joint therapy.

### BRIEF SUMMARY

The present invention is specifically directed towards a type of an enclosure of which its fabric layers are composed of a breathable, porous and moisture resistant type of material of which is utilized and comprised throughout the inner layers of a joint device. Within its open area, the enclosure is formed to receive a therapeutic temperature generating tempered material and or a support type of padding material. The material composition of the present invention is unique in its use of a breathable, porous type of material of which reacts effectively in allowing generated temperature to travel through its fabric composition. The composed material of the enclosure is an ideal catalyst for the use of temperature transfer. The enclosure is quite unique in allowing the wearer the option of utilizing a tempered temperature generating material and or a support type of padding material to be inserted within its open area. Due to the manner in which the enclosure is formed, the wearer is provided with the option of placing the supplemental material within the open area of the enclosure, and the wearer is also provided with the option of wearing the joint device without any inserted supplemental materials placed within the open area of the enclosure.

### BRIEF DESCRIPTION OF THE DRAWING

The invention may take part in the following arrangement, a preferred embodiment of which will be described in detail in this specification, and illustrated in accompanying drawings which form a part hereof.

FIG. 1, is a perspective view of the inner cover layer of a joint device.

FIG. 2, is a side view perspective of an integrated enclosure formed in between the inner layers of a joint device.

FIG. 3, is a top view perspective of an integrated enclosure formed in between the inner layers of a joint device.

FIG. 4, is a side view perspective of an enclosure formed onto the inner layer surface of a joint device.

FIG. 5, is a top view perspective of an enclosure formed onto the inner layer surface of a joint device.

FIG. 6, is a perspective view of an attachable/detachable enclosure.

### DETAILED DESCRIPTION

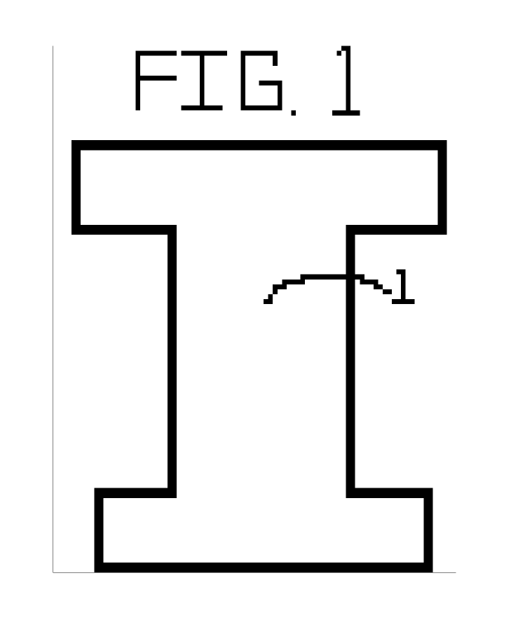

Referring now to the drawings and more particularly to FIG. 1 there is shown an inner layer perspective view of an embodiment of a joint device manufactured using the method of the present invention. In the embodiment shown, the device consists of an inner surface layer (1) of a joint device. The inner surface layer's material composition is of a porous, breathable, moisture-resistant type of material. The material is laid, cut, sewn and stitched in such a manner as to create an integrated enclosure of which is formed in such a manner as would allow the open area of the enclosure to receive a therapeutic, temperature generating, tempered material and/or a cushion support-type of padding material within it's open area, of which will be further illustrated within these specifications. The integrated enclosure may be located within any area of the inner layer of the joint device, and also may be formed to any desired size dimension. There may be any number of enclosures formed, whether single or multiple amounts as would fulfill the intended need of the joint devise.

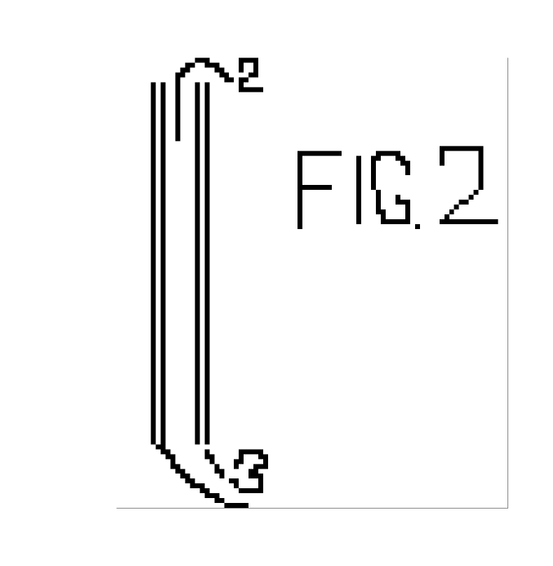

FIG. 2 represents a side view perspective of the embodiment hereby illustrated as the integrated enclosure. The inner layer portion (3) of a joint device is formed in such a manner as to create an enclosure (2) of which is integrated into the inner layer fabric of a joint device. The material composition of the inner layer (3) is of a breathable, porous, moisture-resistant type of material. A therapeutic, temperature generating tempured material and/or a cushion support type of padding material may be inserted into the formed integrated enclosure (2).

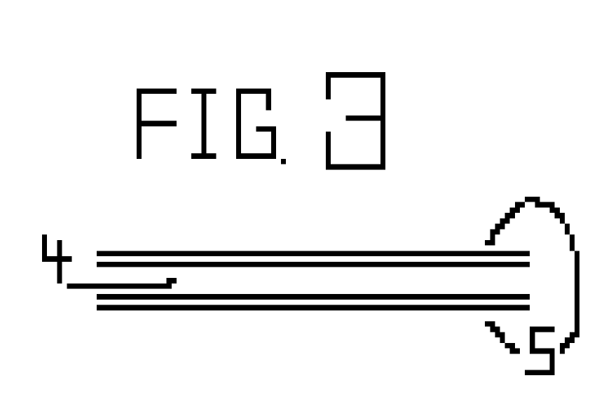

FIG. 3 represents a top-view perspective of the embodiment hereby illustrated as the integrated enclosure. The inner layer portion (5) of a joint device if formed in such a manner as to create an enclosure (4) of which is integrated into the inner lay fabric of a joint device.

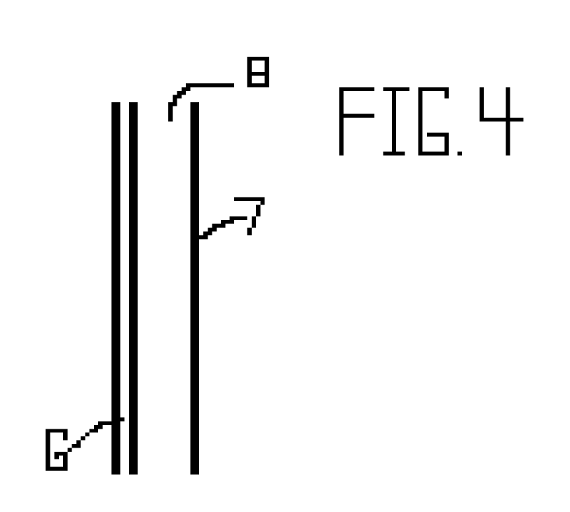

FIG. 4 represents a perspective side view of an enclosure formed onto an inner layer surface (6) of a joint device. A porous, breathable, moisture resistant type of material (7) is overlaid, sewn, but and stitched onto the inner layer surface area (6) of a joint device in such a manner as to form an enclosure (8) of which is formed in such a manner as to allow a therapeutic temperature generating tempured material and/or a cushion support type of padding material to be inserted in to the formed enclosure (8).

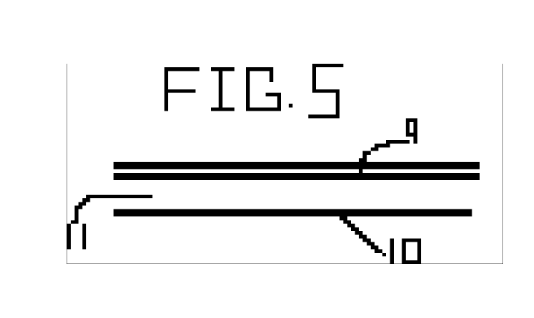

FIG. 5 illustrates a top-view perspective of an enclosure formed onto an inner layer surface area (9) of a joint device. A porous, breathable, moisture resistant type of material (10) is overlaid, sewn, cut and stitched onto the inner layer surface area (9) of which is formed in such a manner as to allow a therapeutic, temperature generating tempured material and/or a cushion support type of padding material to be inserted into the open area (11) of the formed enclosure.

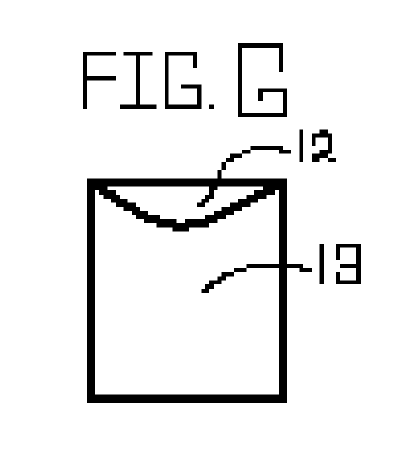

In FIG. 6 is illustrated a perspective view of an attachable/detachable enclosure of which is formed to received a therapeutic, temperature generating, tempured material and/or a cushion support type of padding material within its enclosed area (12). A porous, breathable, moisture-resistant type of material (13) is overlaid, cut, sewn and stitched to create the enclosure. The enclosure is formed in such a manner as to allow a therapeutic temperature generating tempured material and/or a cushioned support type of padding material to be placed into the enclosure's open area (12). The outer surface layer of the enclosure has a velcro-type of adhesive material affixed to its surface of which is used to attach the enclosure to the inner surface area of a joint device of which also possesses a velcro-type of material affixed to it's inner layer surface. Thus is created the embodiment hereby illustrated as the enclosure.

What is claimed is:

1.) A type of enclosure of which is created to incorporate itself throughout the inner layer areas of a joint device and is specifically formed in such a manner as to allow a therapeutic tempered temperature generating material and or a cushion support type of padding material to be inserted into the open area of the enclosure, of which is
Comprised of:
A.) A type of breathable, porous and moisture resistant type of material, i.) wherein the material comprises the inner layers of a joint device, ii.) wherein the material is formed in such a manner as to create a type of enclosure of which is integrated within the inner layer fabric composition of a joint device, of which the enclosure is formed to receive a tempered therapeutic temperature generating material and or a cushion support type of padding material to be placed within its open area.
B.) The enclosure is not limited in scope to any specific location throughout the inner layer areas of a joint device. i.) wherein the enclosure is not limited in scope to any specific sized dimension, ii.) wherein the enclosure is not limited in scope to its numbered multitude, wherein there may be any number of single or multiple enclosures formed throughout the inner layer areas of a joint device by using the method of the present invention.
C.) A type of enclosure of which is created specifically for the purpose of incorporating itself throughout the inner layer areas of a joint device and is specifically formed in such a manner as to receive a therapeutic tempered temperature generating material and or a cushion support type of padding material within its open area.

2.) A type of an enclosure of which is formed onto the inner layer surface area of a joint device and is specifically formed in such a manner as to allow a therapeutic tempered temperature generating material and or a cushion support type of padding material to be placed within the enclosure's open area in order to create the enclosure
Comprised of:
A.) A type of breathable, porous and moisture resistant type of material, i.) wherein the material comprising the composition of the enclosure, ii.) wherein the material is overlaid, cut, sewn and stitched in such a manner as to form a type of enclosure of which comprises the inner layer surface area of a joint device, and is formed in such a manner as to allow a therapeutic tempered temperature generating material and or a cushion support type of padding material to be placed within the enclosure's open area.
B.) The enclosure is not limited in scope to comprising any specific location throughout the inner layer areas of a joint device, i.) wherein the enclosure is not limited in scope to being formed to any specific sized dimension, ii.) wherein the enclosure is not limited in scope to being implemented towards any specific intended use.
C.) A type of enclosure of which is formed onto the inner layer surface area of a joint device of which is formed in such a manner as to receive a cushion support type of padding material within the enclosure's open area.

3.) A type of an enclosure of which attaches and detaches to the inner layer surface areas of a joint device, of which is specifically formed in such a manner as to allow a therapeutic tempered temperature generating material and or a cushion support type of padding material to be received into the opening of the enclosure, of which is
Comprised of:
A.) a type of breathable, porous and moisture resistant type of material, i.) of which the material is laid, cut, sewn and stitched in such a manner as to form an enclosure, ii.) of which the enclosure is specifically formed in such a manner as to allow a therapeutic tempered temperature generating material and or a cushion support type of padding material to be placed into the enclosure's open area. iii) wherein the enclosure's outer surface layer area has a Velcro type of adhesive material affixed to it's surface in such a manner as would consequently allow the enclosure to affix itself onto the inner layer surface of a joint device of which also has a Velcro type of adhesive material affixed to it's inner layer surface area.
B.) A type of an attachable and detachable enclosure is created of which is formed in such a manner as to allow a cushion support type of padding material to be placed into the enclosure's open area.

4.) The enclosures of claims 1-3 wherein the enclosure is formed in such a manner as to allow the enclosures to be incorporated within the inner layer areas of a joint device.
